(19) United States
(12) Patent Application Publication
(10) Pub. No.: US 2022/0079476 A1
(43) Pub. Date: Mar. 17, 2022

(54) MULTICHANNEL HYDRATION MANAGEMENT SENSOR

(71) Applicant: Purdue Research Foundation, West Lafayette, IN (US)

(72) Inventors: Babak Ziaie, West Lafayette, IN (US); Manuel Pimentel Ochoa, Pico Rivera, CA (US)

(73) Assignee: Purdue Research Foundation, West Lafayette, IN (US)

(21) Appl. No.: 17/245,754

(22) Filed: Apr. 30, 2021

Related U.S. Application Data
(60) Provisional application No. 63/018,045, filed on Apr. 30, 2020.

(51) Int. Cl. A61B 5/145; G01N 21/81

(52) U.S. Cl. CPC A61B 5/14517; G01N 21/81

(57) ABSTRACT

A sweat sensor decal may include a first hydrophobic layer comprising a transparent or semi-transparent material and a second hydrophobic layer defining an inlet hole. A substrate layer may be positioned in between the first hydrophobic layer and the second hydrophobic layer such that the inlet hole of the second hydrophobic layer is adjacent to a receiving area on a surface of the substrate layer. The substrate layer comprises a plurality of channels separated by an elongated hole in the substrate layer. The channels may extend away from the receiving area of the substrate layer/Sweat received by the receiving area may be wicked along the channels cause the substrate to change colors.

### CROSS REFERENCE TO RELATED APPLICATIONS

This application claims the benefit of U.S. Provisional Application No. 63/018,045 filed Apr. 30, 2020, the entirety of which is hereby incorporated by reference.

### TECHNICAL FIELD

This disclosure relates to hydration monitoring and, in particular, to decal sensors for hydration monitoring.

### BACKGROUND

Sweat-rate measurements have been conducted using a variety of techniques and devices, with the preferred standard for athletic field use being gravimetry for whole-body sweat rate analysis. More conveniently, localized sweat rates are derived from the mass of sweat collected, or from changes in mass of the filter papers, patches, plastic pouches and ducts. Recently, researchers have developed real time sweat sensing patches with various sampling and sensing modalities that focus on rapid clinical assessments of pH, temperature and various kind of sweat constituents/analytes rather than specifically focusing on massively growing hydration-conscious population for improving commercial/consumer adoption of sweat sensing. Often, they require multiple optical, ampereometric, electrochemicalx or bio impedance based sensors and their measurements are nonlinear with respect to sweat rate due to ion reabsorption phenomenon

For hydration monitoring in strenuous activities such as sports, construction environment, emergency management activities such as firefighting or military applications, low-cost patches with rapid real-time indication and simple read-out methods would be more appropriate. Towards these, various kinds of direct flow rate sensors have recently emerged for application on skin. They include thermal/calorimetric sensors, textile based flow sensors, PDMS based flow sensors and paper based flow sensors (paper is merely used as a passive pump for guiding sweat to some other sensor). These focus more on sweat rate measurements but are either not real time, are bulky, or do not feature sufficiently high temporal resolution or range (or a combination of those) which keeps them out of reach of majority of the global population for day to day usage and makes them difficult to scale up. Moreover, many of them require the wearer to interpret the results through complex steps, potentially diverting focus from other more important tasks (e.g., winning a marathon). Some of the latest PDMS based sensors are driven by the eccrine pressure which can affect the sweat rate itself due to saturation.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates a first example of a sweat sensor decal;

FIG. 2 illustrates an example of a sweat sensor decal with a single channel;

FIG. 3 illustrates an example of a sweat sensor decal with a multi-channel substrate layer;

FIG. 4 illustrates an example of a sweat sensor decal with equidistant and equal width channels;

FIG. 5A-C illustrates a second example of a sweat sensor decal; and

FIG. 6A-C illustrate a third example of a sweat sensor decal.

### DETAILED DESCRIPTION

Sweat-rate measurements have been conducted using a variety of techniques and devices, with the preferred standard for athletic field use being gravimetry for whole-body sweat rate analysis. More conveniently, localized sweat rates are derived from the mass of sweat collected, or from changes in mass of the filter papers, patches, plastic pouches and ducts; but these methods are suited primarily to steady-state conditions and can sometimes result in a progressive occlusion of sweat ducts and sweat suppression. They are also not real time and often require off-board analysis. Several devices based on similar techniques such as Megaduct, Macroduct and Pharmchek have been commercially available since 1990. The water vapor content of a gas can also be measured using a range of methods to quantify trans-epidermal water loss using various methods. The modern hygrometric technique of choice, where both timing and quantification precision is required, rely upon the effect of water vapor on electrical resistance and capacitance¹. However, these methods can be slow, bulky, and can lead to overestimation of sweat rates. Moreover, the trans-epidermal loss is only a small part of total cutaneous loss of water that needs to be replaced for optimum hydration.

Recently, researchers have developed more practical, and real time sweat sensing patches with various sampling and sensing modalities that focus on rapid clinical assessments of pH, temperature and various kind of sweat constituents/analytes rather than specifically focusing on massively growing hydration-conscious population for improving commercial/consumer adoption of sweat sensing. Often, they require multiple optical, ampereometric, electrochemicalx or bio impedance based sensors and their measurements are nonlinear with respect to sweat rate due to ion reabsorption phenomenon. Approaches such as these are suitable for clinical analyses where accurate quantification of dissolved analytes is more important than rapid assessment of perspiration rate and quantification for hydration related information. Because most of these devices target detailed analysis of sweat based on presence of different analytes and are not specifically for sweat rate analysis, they are over designed which renders the system too expensive, bulky, non-real time, complex, unreliable, or non-portable, particularly for applications such as sports. Similarly, some sensors aim to be more accurate by detecting sweat rate using image analysis of commercial sweat collectors; however, these systems are not real-time and require post-processing.

For hydration monitoring in strenuous activities such as sports, construction environment, emergency management activities such as firefighting or military applications, low-cost patches with rapid real-time indication and simple read-out methods would be more appropriate. Towards these, various kinds of direct flow rate sensors have recently emerged for application on skin. They include thermal/calorimetric sensors, textile based flow sensors, PDMS based flow sensors and paper based flow sensors (paper is merely used as a passive pump for guiding sweat to some other sensor). These focus more on sweat rate measurements but are either not real time, are bulky, or do not feature sufficiently high temporal resolution or range (or a combination of those) which keeps them out of reach of majority of the global population for day to day usage and makes them difficult to scale up. Moreover, many of them require the wearer to interpret the results through complex steps, potentially diverting focus from other more important tasks (e.g., winning a marathon). Some of the latest PDMS based sensors are driven by the eccrine pressure which can affect the sweat rate itself due to saturation. One of the biggest challenges in current devices is the personalized nature of sweat rate i.e. the inter-subject variability leading to no standard solution for all users.

To address these and other technical challenges, a sweat sensor decal is provided.

The sweat sensor decal may include a first hydrophobic layer comprising a transparent or semi-transparent material and a second hydrophobic layer defining an inlet hole. A substrate layer may be positioned in between the first hydrophobic layer and the second hydrophobic layer such that the inlet hole of the second hydrophobic layer is adjacent to a receiving area on a surface of the substrate layer. The substrate layer comprises a plurality of channels separated by an elongated hole in the substrate layer. The channels may extend away from the receiving area of the substrate layer, wherein sweat received by the receiving area is wicked along the channels and causes the substrate to change colors.

A technical advantage of the sweat sensor decal described herein includes the ability to monitor sweat flow rate and/or ion concentration using the same fluidic channel. This is accomplished by using colorimetric dyes that respond to ion concentrations. Another advantage is the ability to adapt to various sweat rates by simply changing the thickness and/or geometry of the middle (hygroscopic) layer. Additional and alternative benefits, efficiencies, and improvements over existing approaches are made evident in the description provided herein.

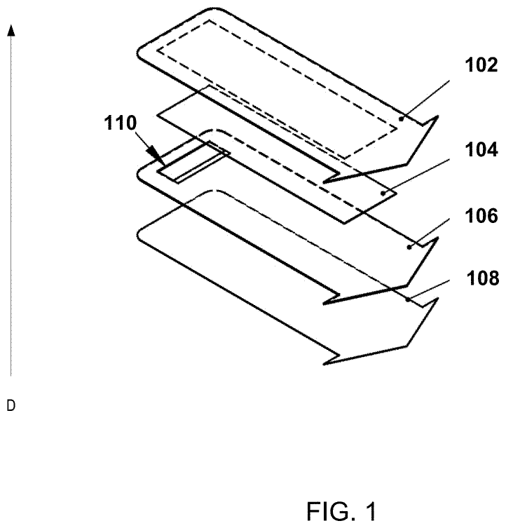

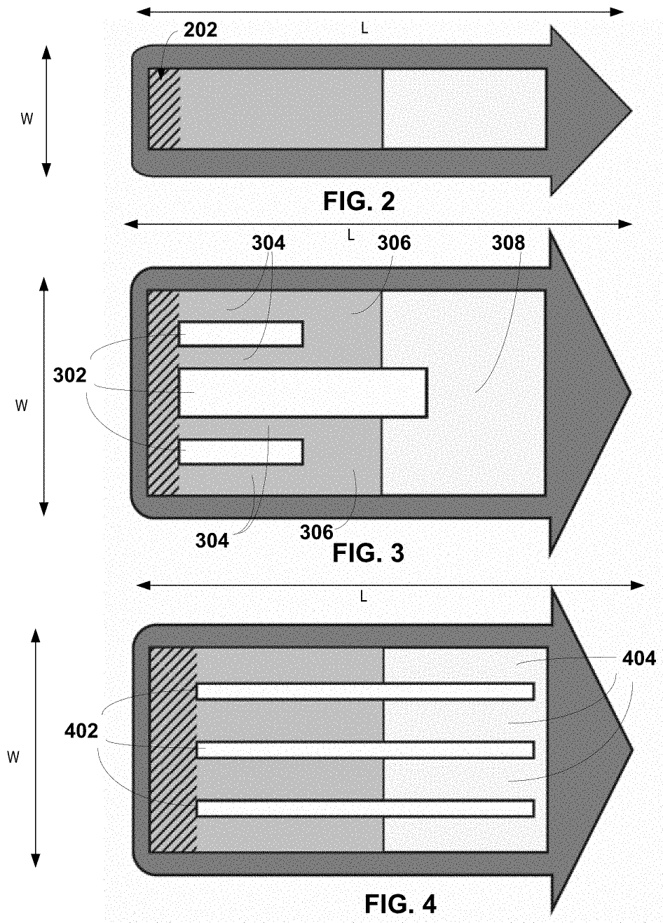

FIG. 1 illustrates a first example of a sweat sensor decal. The system 100 may include a top hydrophobic layer 102, a substrate layer 104, a bottom hydrophobic layer 106 and a liner 108. The top and bottom hydrophobic layers 102,106 may encapsulate the substrate layer 104. The top hydrophobic layer 102 may be positioned further away from a wearer than the bottom layer 104 with respect to a direction D. The top layer 102 may be visible to the wearer during use. Accordingly, the top layer 102 may transparent or semi-transparent and reveal the substrate 104 or other layers beneath the top layer 102. The bottom hydrophobic layer 106 may define an inlet hole 110. The inlet hole 110 may allow moisture to pass through to the substrate layer. A portion of the substrate layer 104 positioned adjacent to the inlet hole 110 may receive the moisture that passes through the receiving hole. For example, an outer surface of the substrate may include a receiving area (hashed area in FIGS. 2-4) that receives the moisture.

The hydrophobic material user in the top and/or bottom layer may include a thin, flexible, waterproof material that conforms to the skin of a subject and is optically transparent at least in the visible wavelength range. For example, the hydrophobic material may include polyurethane, silicone (e.g., PDMS), polyethylene, polystyrene. It should be a material that is impermeable to water (liquid or vapor); the rate of moisture loss across the film should be negligible compared to the rate of sweat wicking into the patch. A urethane is a suitable candidate due to its lower permeability and high flexibility.

The substrate 104 may include hydroscopic material capable of drawing or wicking moisture, such as sweat. For example, the substrate 104 may receive moisture at or near a first end and wick the moisture from the first end of the substrate 104 to a second end opposite of the first. Examples of wicking materials include any cellulosic mesh (e.g., paper, nitrocellulose) or any other material composed of a mesh of hydrophilic fibers or filaments (e.g., glass fiber).

In some examples, the substrate 104 may include the receiving area 202. This area can be of various dimensions and can contain a pattern of openings to control the flowrate of sweat from the skin. Alternatively or in addition, the receiving area may instead be a hole in the substrate layer that overlaps the inlet port 110 (See FIG. 1).

In some examples, the substrate 104 may change color in response to receiving moisture. For example, the substrate 104 may include a colorimetric sensor or dye that activates in response to contact with certain analytes in sweat. In some embodiments, the die or colorimetric sensor may include KMnO₄ or CoCl₂ (or CoCl₂*2H₂O), which changes from blue to magenta when wetted) may be deposited on the substrate. The die may be deposited via a stamping, or some other suitable technique. In some examples, the die may be included on an entity of the substrate such that the portion exposed to sweat changes color while the portion not exposed remains neutral.

When worn, the bottom hydrophobic layer of the patch is in contact with or immediately adjacent to the subject's skin. The hydrophobic layer may be coated with a skin-suitable adhesive that allows the patch to stick to the subject.

Before usage, the skin adhesive is protected by a liner 108 (e.g., a film with low surface energy such as wax paper, parchment paper, or polyethylene-coated paper) which is peeled off and discarded immediately before donning the patch.

FIGS. 2-4 illustrate various examples sensors with different substrates. FIG. 2 illustrates an example of a sweat sensor decal with a single channel. The receiving area 202 (indicated with hashed lines in FIG. 2) may be positioned on a first end of the sensor. The channel may extend away from the receiving area toward a second end of the sensor. The substrate may changed colors as sweat is wicked along the substrate.

FIG. 3 illustrates an example of a sensor with a multi-channel substrate. The substrate layer 104 may have a plurality of elongated holes 302 that define a plurality of channels 304 in the substrate layer. The holes 302 may be defined by the substrate layer 104. At least one of the holes 302 may extend away from the receiving area. Alternatively, multiple holes may extend away from the receiving area along the length L of the sensor. Depending on the location and dimensions of the channels, the channels may vary in width and length. Width w is a dimension perpendicular to length L. Length extends between the first end and send end of the sensor.

The channels 304 may be portions of the substrate that are separated by the holes, with respect to the width of the sensor. Some of the holes may be shorter in length than other channels. In addition, some of the holes may be shorter in length then others, causing some channels to join with others channels to form a merged channel. In some examples, merged channels may be further defined by one or more holes, and merged channels may join with other merged channels.

By way of example, the substrate may include a first plurality of channels 304 separated by a plurality of elongated holes 302, respectively. At least one of the elongated holes may be longer and/or wider than then other holes. The longer hole may be positioned in between the shorter holes. In some examples, the longer hole may extend beyond the shorter holes and the channels may merge into one or more intermediate merged channels 306. Where the longer hole ends, the one or more intermediate merged channels 306 may merge into one or more tertiary merged channel, and so on. In some examples, the a full channel 308 may extend the entire width of the substrate and not be separated by any holes.

Said another way, the substrate may include a first pair and second pair of channels. The first pair of channels may be separated by a first elongated hole. The second pair of channels may be separated by a second elongated hole. The first pair of channels may merge at a first merging location where a first merged channel is formed at an end of the first elongated hole. The second pair of channels may merge at a second merging location where a second merged channel is formed at the end of the second elongated hold. The first merged channel and the second merged channel may be separated by a third elongated hole. The first merged channel and the second merged channel may merge at a third merging location where a third merged channel is formed at an end of the third elongated hole. In some examples, this pattern could repeat until a final merged channel is formed that encompasses the width of the substrate.

While FIG. 3 illustrates an example with various merging locations, additional or fewer merge locations are possible, depending on the number, length, and width of the elongated holes. Furthermore, the length and width of the channels may depend on the number, length, and width of the holes.

FIG. 4 illustrates an example of a sweat sensor decal with equidistant and equal width channels. The equidistant channels may be defined between a plurality of elongated holes in the substrate that are equitant. The holes may have the same dimensions and positioned at locations equitant apart such that the dimensions of each channel are the same. The channels 404 may extend from the receiving area at a first end of the substrate layer to a second end of the substrate layer opposite the first end. In some examples, the channels may merge into a final channel that extends the entire width of the substrate layer, as illustrated in FIG. 4. Alternatively the channels may bento merge and extend to the end of the substrate layer.

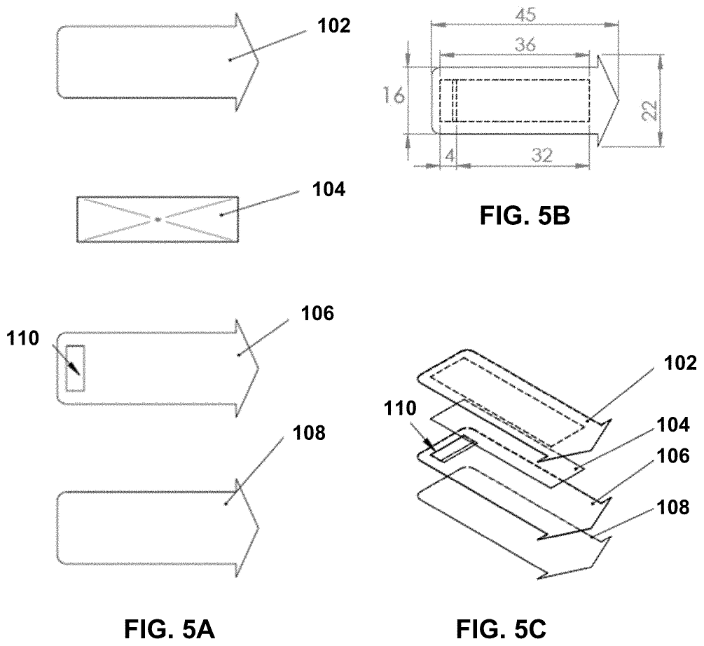

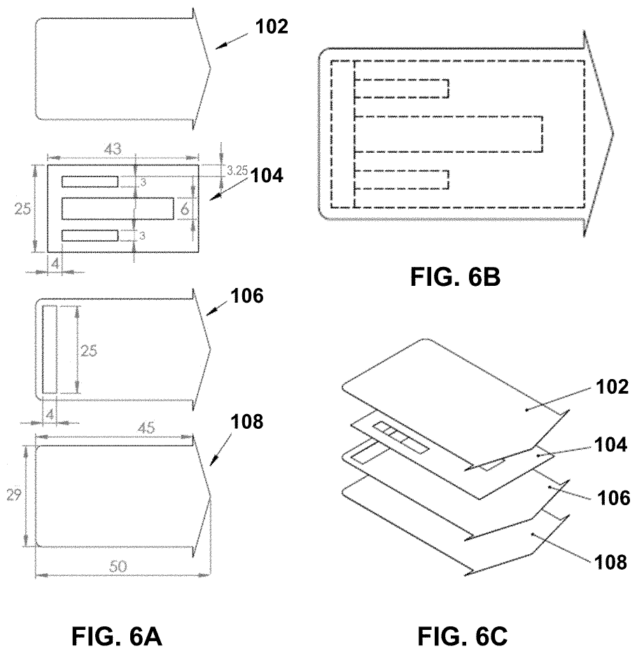

FIGS. 5A-C illustrates a second example of the sensor. FIG. 5A illustrates each layer of the sensor. FIG. 5B illustrates a top view of the sensor with example dimensions. FIG. 5C illustrates an isometric perspective of the sensor. FIG. 6A-C illustrate a third example of the sensor. FIG. 6A illustrates each layer of the sensor. FIG. 6B illustrates a top view of the sensor with example dimensions. FIG. 6C illustrates an isometric perspective of the sensor.

It should be appreciated that the dimensions and number of holes/channels are provided as examples but may vary in implementation. The patches may be fabricated using three layers (plus a liner). The substrate layer may include a paper and may be pre-loaded with a chemical that is sensitive to moisture, pH, or other analytes in sweat. Examples of various chemicals that may be pre-loaded include silver chloroanilate, 2-7-dichlorofluorescein, cobalt hexachloride, powdered dye that increases in visibility when wet (e.g., KMnO₄), or any colorimetric ion sensor. The patches can be fabricated on a sheet-to-sheet or roll-to-roll manufacturing setup which includes a laser system to produce mass-customized patches by changing the dimensions of a cut on one layer.

A second action may be said to be “in response to” a first action independent of whether the second action results directly or indirectly from the first action. The second action may occur at a substantially later time than the first action and still be in response to the first action. Similarly, the second action may be said to be in response to the first action even if intervening actions take place between the first action and the second action, and even if one or more of the intervening actions directly cause the second action to be performed. For example, a second action may be in response to a first action if the first action sets a flag and a third action later initiates the second action whenever the flag is set.

To clarify the use of and to hereby provide notice to the public, the phrases “at least one of <A>, <B>, . . . and <N>” or “at least one of <A>, <B>, <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations.

What is claimed is:

1. A skin-based sweat sensor decal comprising:
a first hydrophobic layer comprising a transparent or semi-transparent material;
a second hydrophobic layer defining an inlet hole; and
a substrate layer positioned in between the first hydrophobic layer and the second hydrophobic layer such that the inlet hole of the second hydrophobic layer is adjacent to a receiving area on a surface of the substrate layer, wherein the substrate layer comprises a plurality of channels separated by an elongated hole in the substrate layer, wherein the channels extend away from the receiving area of the substrate layer, wherein sweat received by the receiving area is wicked along the channels and causes the substrate to change colors.

2. The sweat sensor of claim 1, wherein the channels extend toward a merged channel of the substrate layer at an end of the elongated hole.

3. The sweat sensor of claim 1, wherein the channels are separated by respective elongated holes in the substrate.

4. The sweat sensor of claim 3, wherein the respective elongated holes are parallel.

5. The sweat sensor of claim 3, wherein the channels are parallel.

6. The sweat sensor of claim 3, wherein the channels comprise a first pair and second pair of channels, the first pair of channels separated by a first elongated hole and the second pair of channels separated by a second elongated hole, the first pair of channels merging at a first merged channel of the substrate layer, the second pair of channels merging at a second merged channel of the substrate layer, wherein the first merged channel and the second merged channel are separated by a third elongated hole.

7. The sweat sensor of claim 6, wherein the first merged channel and the merged channel merge at a third merged channel.

8. The sweat sensor of claim 3, wherein the respective elongated holes are a same length.

9. The sweat sensor of claim 1, wherein the channels are a same length.

10. The sweat sensor of claim 1, wherein the channels are a same width.

11. The sweat sensor of claim 1, wherein an outer surface of the hydrophobic layer comprises an adhesive configured to adhere to skin.

12. The sweat sensor of claim 11, further comprising a liner applied to the outer surface of the second hydrophobic layer.

13. The sweat sensor of claim 1, wherein the substrate is configured to change color in response contact with sweat.
